(19) United States
(12) Patent Application Publication
(10) Pub. No.: US 2023/0414880 A1
(43) Pub. Date: Dec. 28, 2023

(54) KIT FOR THE SUCCESSIVE AND COAXIAL INJECTION OF A MEDICAMENT

(71) Applicant: GEM S.R.L, Viareggio (IT)

(72) Inventors: Giovanni FACCIOLI, Sommacampagna (IT); Renzo SOFFIATTI, Sommacampagna (IT); Lorenzo TESSARI, Peschiera Del Garda (IT); Mirko TESSARI, Peschiera Del Garda (IT)

(21) Appl. No.: 18/254,807

(22) PCT Filed: Jun. 30, 2021
(86) PCT No.: PCT/IB2021/055855

§ 371 Date: May 26, 2023

(30) Foreign Application Priority Data
Nov. 27, 2020 (IT) 102020000028709

(51) Int. Cl. A61M 5/315

(52) U.S. Cl. CPC A61M 5/31596; A61M 5/31511

(57) ABSTRACT

A kit for subsequent injection and/or subsequent insertion of at least one medical substance in the fluid or liquid state and/or of at least one element in a human body injection or insertion site. The kit includes a first syringe element and at least one second syringe element. The first syringe element includes an internally hollow cylindrical body, a plunger and a first needle. The plunger is suitable to slide inside the cylindrical body in use. The at least one second syringe element includes a piston and a second needle. The at least one medical substance is inserted into the cylindrical body and the first syringe element is suitable to house, at least partially therein, the at least one second syringe element or the second needle is suitable to pass through the plunger in use.

### TECHNICAL FIELD

The present invention relates to a kit for the subsequent injection and/or subsequent insertion of substances and/or elements in the human body in a coaxial manner

More particularly, the present invention relates to a kit for the subsequent injection and/or subsequent insertion of substances and/or elements into the human body, such as for example the same substance or two different substances at successive times in the same injection site or a substance and a different element, such as a further device, in successive times at the same injection site.

### BACKGROUND

In the medical field, it is very often useful to insert, for example inject, a same substance or more substances into the same implantation site at successive times.

In order to do this, for example there arises the need to inject a first substance into a given site and then to be able to inject the second substance (or in any case to carry out the second injection) exactly into the same site.

Clearly, this is a very difficult operation, which requires great precision for its success, especially when the area to be injected is small in size.

An example of such a method is that relating to the treatment of varices or varicose veins, wherein a method for the reduction thereof provides for the injection—into the vein to be treated—of a surfactant substance (or surfactant sclerosant such as for example sodium tetradecyl sulphate known by the trade name Fibro-Vein® or Trombovar® or the oxy-polyethoxy dodecane known by the trade name Atossisclerol® or Sotradecol®) which causes the vein to contract and subsequent injection—using the same needle and therefore exactly at the same site of the human body—of an adhesive substance which allows to seal the bloodstream by gluing the inner walls of the venous tract of interest.

Such technique is also known as sclerotherapy with “mousse” or as “scleromousse” (tourbillon technique). This technique is officially identified by the ruling issued by AIFA (Italian Medicines Agency) on Fibro-Vein® in 2015 and on Atossisclerol® in 2019.

The surfactant sclerosant allows to increase, with the same drug concentration, the contact of the scleros ant with the endothelium of the vein.

In such technique, two disposable plastic syringes and a special connector, still made of disposable plastic, are used to mix (by passing from one syringe to another by means of the connector), the biocompatible air or gases and the surfactant sclerosant, at the selected doses. A dense and compact mousse, which can be injected at the prescribed doses, comprising microbubbles of gas and drug, is obtained in a few seconds and through about ten steps. The size of such microbubbles may depend on several factors, such as the type of connector, the syringes used, etc . . . , which are linked to the specific requirements and techniques of manufacture.

It is a procedure that requires great dexterity and attention by the surgeon so as to ensure the success of the operation.

Such procedure is also required in other medical fields, as mentioned, for inserting two different substances one after the other or the same substance at two different times into the human body.

There is therefore a need for a device or kit that allows to carry out the actions indicated above—and also others that generally meet the same principle—in an easier and safer manner

### SUMMARY

A kit for subsequent injection and/or subsequent insertion of at least one medical substance in the fluid or liquid state and/or of at least one element in a human body injection or insertion site. The kit includes a first syringe element and at least one second syringe element. The first syringe element (includes an internally hollow cylindrical body, a plunger and a first needle. The plunger is suitable to slide inside the cylindrical body in use. The at least one second syringe element includes a piston and a second needle. The at least one medical substance is inserted into the cylindrical body and the first syringe element is suitable to house, at least partially therein, the at least one second syringe element or the second needle is suitable to pass through the plunger in use.

### BRIEF DESCRIPTION OF THE DRAWINGS

Further characteristics and advantages will be more apparent from the detailed but non-exclusive description of a kit for subsequent injection and/or subsequent insertion of substances and/or elements into the human body, provided by way of non-limiting example in the attached drawings, wherein:

FIG. 1 is a rear perspective view of a kit according in a version in which the two components of the kit are separated from each other;

FIG. 2 is a lateral view of the kit of FIG. 1;

FIG. 3A is an only partially sectional lateral view of the kit of FIG. 2, taken along the plane of line A-A;

FIG. 3B is a rear perspective and only partially sectional view of the kit of FIG. 2, taken along the plane of line A-A;

FIG. 4 is a lateral view of the kit according to the preceding figures according to an operative step;

FIG. 5A is an only partially sectional lateral view of the kit of FIG. 4 taken along the plane of line A-A;

FIG. 5B is a rear perspective and only partially sectional view of the kit of FIG. 4, taken along the plane of line B-B;

FIG. 6 is a rear perspective view of the kit wherein the two components of the kit are in the assembled version;

FIG. 7 is a lateral view of the kit of FIG. 6;

FIG. 8A is an only partially sectional lateral view of the kit of FIG. 7, taken along the plane of line C-C;

FIG. 8B is a rear perspective and only partially sectional view of the kit of FIG. 7, taken along the plane of line C-C;

FIG. 9 is a view of an enlarged detail and a sectional view of the kit of FIG. 8A;

FIG. 10A is a partially sectional lateral view of a version of the kit for the subsequent injection and/or subsequent insertion of substances and/or elements;

FIG. 10B is a sectional lateral view of the kit of FIG. 10A, taken along the plane of line D-D;

FIG. 10C is a view of an enlarged detail of the kit of FIG. 10A;

FIGS. 11A and 11B are two perspective views of the detail of FIG. 10C, respectively in assembled and disassembled version with a further element of the kit of FIG. 10A;

FIG. 12A is a partially sectional front perspective view of a further version of the kit for the subsequent injection and/or subsequent insertion of substances and/or elements;

FIG. 12B is a lateral view from the side of the kit of FIG. 12A;

FIG. 12C is a top lateral view of the kit of FIG. 12A;

FIG. 12D is a perspective view of an enlarged detail of the kit of FIG. 12A;

FIGS. 12E and 12F are respectively lateral and perspective views of an element of the kit of FIG. 12A;

FIG. 13 is an exploded view of the kit;

FIG. 14 is an exemplifying lateral view of a kit for the subsequent insertion of at least one substance and at least one element, in which the second component of the kit is represented in two different operative versions;

FIG. 15 is a lateral view of a further version of a kit for the subsequent injection of substances in which the two components of the kit are combined together,

FIG. 16 is a lateral view of a further version of a kit.

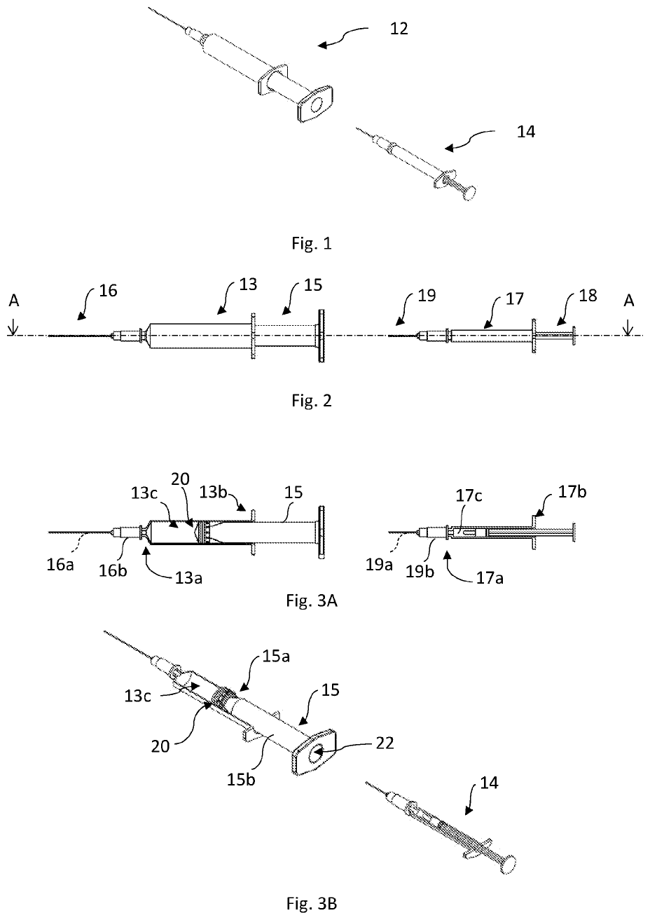

### DETAILED DESCRIPTION

The object of the current disclosure is to improve the state of the art in the field of devices for the injection and/or insertion of substances and/or elements into the human body.

In the scope of such technical task, an object of the current disclosure is to provide a kit for the subsequent injection and/or subsequent insertion of substances and/or elements which is practical to use and cost-effective.

A further object of the current disclosure is to provide a kit for the subsequent injection and/or the subsequent insertion of substances and/or elements that can guarantee exactly the same injection/insertion site while having to carry out these operations in successive times with respect to each other.

Still another object of the current disclosure is to provide a kit for the subsequent injection and/or subsequent insertion of substances and/or elements that can be used by medical personnel simply and quickly.

With reference to the attached figures, a kit for the subsequent injection and/or the subsequent insertion of substances and/or elements into the human body is indicated in its entirety with reference number 10.

According to the present disclosurein, the expression “injection” and the expression “insertion”, as well as the expression “substance” and the expression “element” will be used as synonyms, unless specified otherwise.

Furthermore, unless otherwise specified the expression “fluid” is used to indicate a liquid substance, gaseous substance, plasma, etcetera, or a combination thereof.

The kit 10, in a first version thereof, comprises a first syringe element 12 and at least one second syringe element 14.

The expression “subsequent injection” or “subsequent insertion” into a site of the human body is used to indicate that the first syringe element 12 is adapted in use to determine the site of injection and/or insertion into the human body and the second syringe element 14 is suitable in use to inject or insert at least one substance or at least one element into the same injection/insertion site determined by the first syringe element. Therefore, the expression “subsequent” means that it comes after the injection and/or the insertion or suctioning carried out by the first syringe element 12, as will be better explained hereinafter.

According to the version illustrated in the figures exclusively by way of example, the first syringe element 12 comprises a 5 ml syringe while the second syringe element 14 comprises a 2 ml syringe.

Naturally, different capacities for the first 12 and second 14 syringe element may also envisaged. However, the first syringe element 12 has a greater capacity than the second syringe element 14.

Furthermore, the first syringe element 12 is capable of housing the second syringe element 14 at least partially therein.

In at least one embodiment of the current disclosure, the kit 10 exclusively consists of the first syringe element 12 and of the second syringe element 14, obviously besides the substances, for example medical substances, and/or elements which can be contained therein and which can be inserted into the human body.

As a matter of fact, the first syringe element 12 and the second syringe element 14 are suitable to contain at least one substance and/or at least one element to be inserted into the human body, such as for example a substance in the fluid or liquid state to be injected into the human body, an element 30 comprising an optical fibre device, a laser fibre device, a Radio Frequency (RF) device or antenna, a device capable of transporting and/or dispensing steam or other suitable gas, for example being tubular-shaped, a device capable of generating heat and/or radiation, for example in the infrared spectrum, or microwaves, etcetera.

In some embodiments, the substance contained in the first syringe element 12 is for example a medical substance such as for example a biocompatible surfactant substance (such as, for example, a surfactant sclerosant such as sodium tetradecyl sulphate known by the trade name Fibro-Vein® or Trombovar® or oxy-polyethoxy dodecane known by the trade name Atossisclerol® or Sotradecol® etcetera) while the substance contained in the second syringe element 14 is for example a medical substance such as a biocompatible adhesive agent, for example based on cyanoacrylate, such as for example the one known by the trade name Loctite-Attak.

The maintenance of the injection/insertion position is extremely important also due to the fact that, for example as regards Loctite-Attak but also for other medical substances or adhesive agents, it acts by gluing only if the surfaces to be joined are in contact with each other, and therefore only after the inner walls of the veins to be treated (should such kit be used as an alternative for the sclerotherapy procedure indicated above) were collapsed by means of the first medical substance or by means of the surfactant agent. In a further version, the substance contained in the first syringe element 12 is the same as that contained in the second syringe element 14, possibly having different dose and/or different density or the same density.

In some embodiments, the substances present in the first 12 and/or second 14 syringe element may be biocompatible cementing elements.

In some embodiments, the first syringe element 12 may be used to suction a biological liquid present at a given surgical site while the second syringe element 14 can be used to inject, exactly at the suctioning site, the substance contained therein.

In this case, the second syringe element 14 can be provided with a second extra-long needle 19 capable of piercing (as better described hereinafter and as visible in FIG. 16) the plunger 15 and/or the capsule or gasket 20 of the head 15a of the plunger 15 and overcome the possible liquid suctioned by the first syringe element 12. Therefore, a bicomponent resin such as bone cement, possibly based on liquid poly(methyl methacrylate) (PMMA) can be inserted into the second syringe element 14 and injected into the vein or into the injection site where, after a few minutes, hardening, it blocks any blood outflow.

Getting into the detail of some embodiments, the first syringe element 12 comprises an internally hollow cylindrical body 13, a plunger 15 (or first piston 15) and a first needle 16. The plunger 15 slides inside the cylindrical body 13, along a longitudinal direction determined precisely by the cylindrical body 13 and/or by a longitudinal axis of the first syringe element 12.

As a matter of fact, the first syringe element 12 extends along a longitudinal axis (first longitudinal axis) which can also be the symmetry axis thereof.

The second syringe element 14 extends along a longitudinal axis (second longitudinal axis) which can also be the symmetry axis thereof and which—in use—coincides with the longitudinal axis of the first syringe element 12.

Similarly, the second syringe element 14 comprises an internally hollow cylindrical side wall 17, a piston 18 (or second piston 18) and a second needle 19.

The first needle 16 and the second needle 19 are internally hollow and/or comprise respectively a first inner lumen 16a and a second inner lumen 19a.

There is also present a first connection element 16b between the first needle 16 and a first end 13a of the cylindrical body 13 to connect, possibly removably, the first needle 16 and the cylindrical body 13 of the first syringe element 12.

Similarly, there may be present a second connection element 19b between the second needle 19 and a tip 17a of the cylindrical side wall 17 to connect, possibly removably, the second needle 19 and the cylindrical side wall 17 of the second syringe element 14. The first connection element 16b and/or the second connection element 19b may comprise a Luer lock.

The cylindrical body 13 determines a first end 13a, to which the first needle 16 is constrained, possibly removably, and a second end 13b, opposite the first end 13a. The second end 13b is the open end of the cylindrical body 13, from which the cavity 13c inside the cylindrical body 13 can be accessed in use.

On the other hand, the first end 13a is an end closed by the first needle 16 but placed in fluid communication with the cavity 13c and with the inner lumen 16a of the first needle 16.

The first end 13a has, in some embodiments, a tubular shape, for example cylindrical or truncated cone, having a cross-section, for example circular, and it is fitted into a special seat for housing the first connection element 16b.

The plunger 15 hermetically slides inside the cylindrical body 13 and/or inside the cavity 13c thereof.

The cylindrical side wall 17 comprises a tip 17a, to which there is constrained, possibly removably, the second needle 19, and a distal end 17b, opposite to the tip 17a. The distal end 17b is the open end of the cylindrical side wall 17, from which the space 17c inside the cylindrical side wall 17 can be accessed in use.

On the other hand, the tip 17a is an end closed by the second needle 19 but placed in fluid communication with the space 17c and with the inner lumen 19a of the second needle 19.

The tip 17a has, in some embodiments, a tubular shape, for example cylindrical or truncated cone, for example having a circular cross-section, and it is fitted into a special seat for housing the second connection element 19b.

Therefore, both the first end 13a and the tip 17a have—therein—a connection channel 13a′, 17a′ respectively with the first lumen 16a of the first needle 16 and with the second lumen 19a of the second needle 19, as well as with the cavity 13c and with the space 17c.

The piston 18 slides, for example hermetically, inside the space 17c.

Contained in the cavity 13c and/or the space 17c is at least one substance to be injected into the human body and/or the at least one element 30 to be inserted into the human body (as will be described in greater detail hereinafter).

The at least one substance and/or the at least one element flows through the inner lumen 16a of the first needle 16 and/or through the inner lumen 19a of the second needle 19, pushed respectively by the plunger 15 and/or by the piston 18.

While the piston 18 is generally, in some embodiments, of the known type, the plunger is peculiar.

In particular, the plunger 15 has a head 15a, which is suitable in use, when fully inserted into the cavity 13c and/or into the cylindrical body 13, to internally come into contact with the first end 13a of the cylindrical body 13.

The head 15a is faced toward the first needle 16.

In addition, the plunger 15 has a stem 15b. The stem 15b is internally hollow and/or it comprises an internal channel 22. The channel 22 is a channel passing from the head of the plunger 15 up to an end part 21 thereof.

The plunger 15 and/or the head 15a of the plunger 15 comprises a capsule or gasket 20. The capsule or gasket 20 is generally made of rubber and/or it has a substantially conical shape or in any case corresponding to the inner area of the cylindrical body 13 at the first end 13a thereof.

The capsule or gasket 20 is hermetically sealed in the sense that it allows the sliding of the plunger 15 inside the cavity 13c of the cylindrical body 13 but, at the same time, it prevents the solution contained in the cavity 13c from flowing out from the side of the capsule or gasket 20.

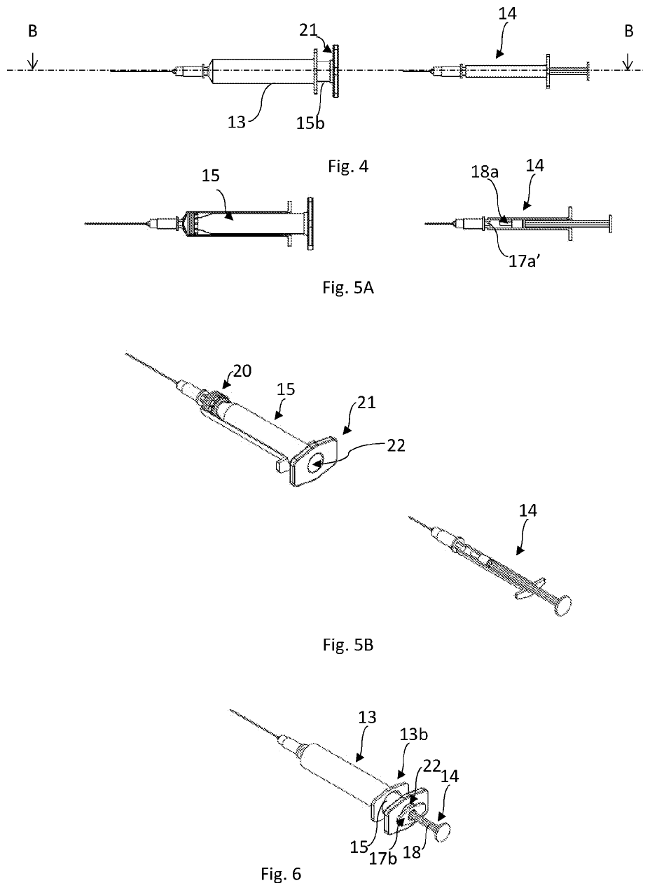

When the plunger 15 is fully inserted into the cavity 13c of the cylindrical body 13, the head 15a and/or the capsule or gasket 20 is internally in contact with the first end 13a of the cylindrical body 13 and the solution inserted into the cavity 13c is made to fully flow out of the first needle 16. Such conformation is for example visible in FIGS. 4, 5A and 5B.

In this case, the stem 15b is almost fully housed inside the cavity 13c, except for the end part 21 part. As observable from such figures, the end part 21 is shaped so as to allow it to be gripped and/or moved, for example by two operator fingers, and for example it is flanged.

As mentioned, the stem 15b of the plunger 15 has a hollow internal channel 22.

Such internal channel 22 is arranged coaxially with the stem 15b and/or it is positioned parallel and/or coaxial with respect to the longitudinal direction of the first syringe element 12 and/or to the sliding direction of the plunger 15.

Such internal channel 22 is shaped so as to allow the housing of the second syringe element 14 therein and/or in the recess determined by it. The conformation of the internal channel 22 is therefore suitable to allow the insertion of the second syringe element 14 thereinto and the subsequent exit.

In particular, in some embodiments, the internal channel 22 has a conformation corresponding to and/or complementary to that of the second syringe element 14, but slightly larger in size specifically so as to allow the movement of the latter and/or the housing thereof, possibly removable.

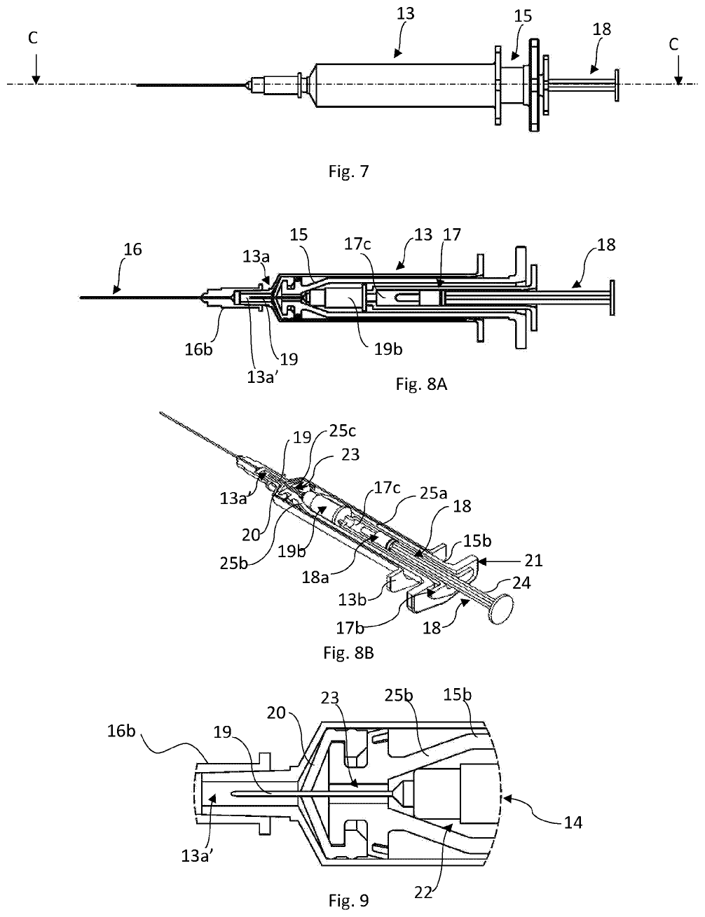

The dimensions of the internal channel 22 do not exceed excessively those of the second syringe element 14 because the internal channel 22 must act as a guide for the insertion and/or housing and/or exit of the second syringe element 14. As a matter of fact, in use, as visible for example in FIG. 8A, when the second syringe element 14 is housed in the first syringe element 12, in order to carry out the operation of the kit, the second needle 19 penetrates at least partially into the connection channel 13a′ of the first end 13a of the cylindrical channel 13 or into the connection element 16b. It is therefore evincible that in order to allow such insertion correctly, the second syringe element 14 must be fittingly housed inside the internal channel 22 of the plunger 15 of the first syringe element 12. Such version is also illustrated, for example, in FIGS. 6 and 7.

In order to penetrate into the connection channel 13a′ and/or into the first connection element 16b, the second needle 19 pierces the capsule or gasket 20 of the stem 15b of the plunger 15.

As observable for example in such figures, housed inside the channel 22 are the second needle 19 (at least partially), the second connection element 19b, at least part of the cylindrical side wall 17 and thus of the piston 18 which is contained therein.

At the joint with the capsule or gasket 20, the head 15a and/or the respective end of the stem 15b of the plunger 15, in some embodiments, may have a gap or a hole 23 which allows the second needle 19 to pass and reach the capsule or gasket 20 to pierce the latter and continue the movement thereof toward the first needle 16.

This is also visible in the detail of FIG. 9.

The sealing of the plunger 15 is in any case ensured by the presence of the capsule or gasket 20.

When the first syringe element 12 and the second syringe element 14 of the kit 10 are assembled in this manner, it is possible to press the piston 18 of the second syringe element 14 and to let the substance contained in the space 17c flow out through the second needle 19 and then through the first needle 16. The injection of the substance contained in the second syringe element 14 (or second substance) is thus ensured exactly at the point of injection of the substance contained in the first syringe element 12 (or first substance) or at the point where the latter suctioned a possible biological liquid.

In the latter case, as mentioned above, the plunger 15 will be in a position moved away from the first end 13a of the cylindrical body 13 of the first syringe element 12 and therefore, in order to reach the first needle 16, the second needle 19 could have an adequate length (as observable in FIG. 16) or there could be a system, for example a suctioning system, for eliminating the liquid suctioned by the first needle 16 into the human body, so as to be able to approach the head 15a of the plunger 15 toward the first end 13a of the first syringe element 12, and thus allowing the second needle 19 to reach in proximity of the first needle 16 and carry out the injection of the substance contained in the second syringe element 14.

The piston 18 of the second syringe element 14 is provided with a stem 24. The stem 24 can be solid, possibly of the conventional type, when it has the sole purpose of passing through the space 17c and to push out the substance present in the second syringe element 14.

Alternatively, the stem 24 can be hollow and house the at least one element 30 therein, as will be better described hereinafter.

The head 18a of the piston 18 has a conformation substantially complementary to the internal one of the tip 17a of the cylindrical side wall 17 and/or of the connection channel 17a′ of the tip 17a in order to press and make all the substance contained in the second syringe element 14 and/or in the space 17c flow out from the second needle 19.

As observable in the figures, and in some embodiments, the plunger 15 and/or the stem thereof has a substantially tubular-shaped portion 25a with a circular cross-section, with which there is associated an internally hollow cone or truncated cone portion 25b, at the head 15a. The base of the cone or truncated cone portion 25b coincides with one of the bases of the substantially tubular-shaped portion 25a. The internal channel 22 of the stem 15 is formed both by the inner space of the substantially tubular-shaped portion and by the inner space of a cone or truncated cone portion 25b. The latter is particularly suitable to fittingly house the second connection element 19b of the second syringe element 14.

On the other hand, at the apical area of the cone or truncated cone portion 25b, there is present a discoidal portion 25c, which acts as a base for supporting and constraining the capsule or gasket 20.

Both the apical area of the cone or truncated cone portion 25b and the discoidal portion may comprise the gap or hole 23 for the through-passing of the second needle 19.

This gap or hole 23 can be useful to facilitate the through-passing of the second needle 19 given the nature of the material the stem of the plunger 15 is made of.

As a matter of fact, the first syringe element 12 is substantially fully made (except for the first needle 16 and the capsule or gasket 20) of a plastic material resistant to deformation, in order to carry out the substance injection or suctioning function thereof.

The first needle 16 and the second needle 19 are made of a metal material. The capsule or gasket 20 is made of a resistant but deformable material, and therefore suitable to allow the sliding of the plunger 15 and the possibility of being pierced by the second needle 19.

Even the second syringe element 14 is substantially fully made (except for the second needle 19 and a possible capsule or gasket) of a plastic material resistant to deformation, in order to carry out the substance injection or suctioning function thereof.

The shape of the capsule or gasket 20 is substantially complementary to the inner shape of the first end 13a of the cylindrical body 13, except, in some embodiments

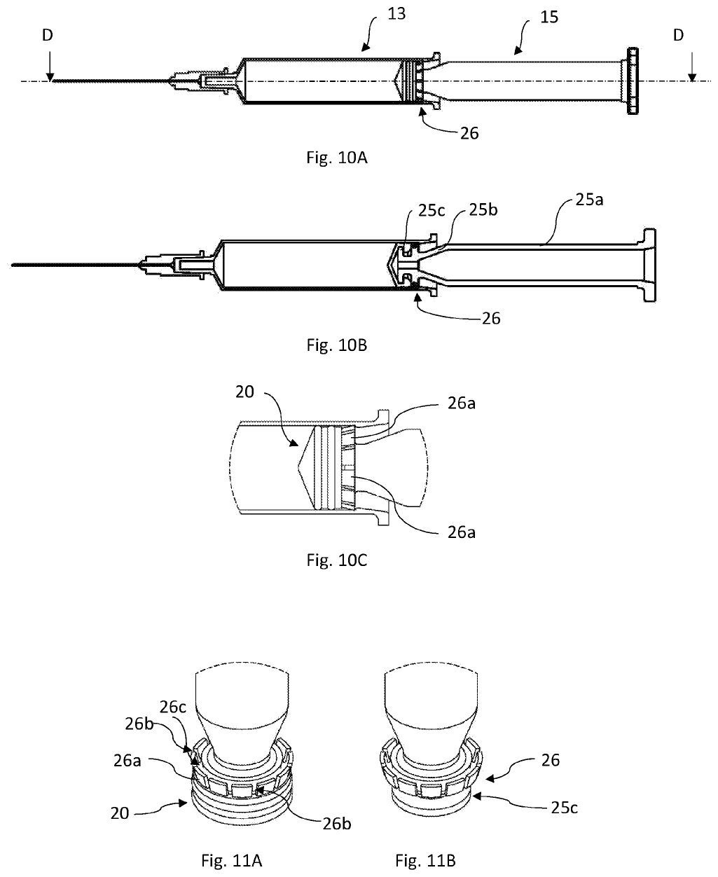

In some embodiments, as observable in the FIGS. 10A, 10B, 10C, 11A and 11B, the plunger 15 of the first syringe element 12 has a stop element 26. Such stop element 26 is an anti-removal element for the plunger 15, which thus cannot be erroneously removed from the cylindrical body 13 of the first syringe element 12.

As observable in the detail of FIG. 11A and 11B, such stop element 26 is substantially annular-shaped. It comprises a plurality of fins 26a, potentially flexible, arranged at the joint between the capsule or gasket 20 and the head 15a of the plunger 15. Such plurality of fins 26a is arranged in an annular manner around the head 15a. Each fin 26a extends from the head 15a of the plunger 15 toward the external with respect to the latter and it is faced toward the end part 21 of the plunger 15.

The plurality of fins 26a provides for that in such stop element 26 each fin is separated from the adjacent fin. In this manner, a space or gap 26b is created between one fin and the other and this allows the fins 26a to bend and/or move and/or change position should the plunger 15 be moved toward the first end 13a or toward the second end 13b of the cylindrical body 13.

In particular, should the plunger 15 be pressed toward the first end 13a of the cylindrical body 13, the fins 26a will be forced toward the stem 15b allowing the sliding of the plunger 15 in such direction; therefore, on the other hand, when the plunger 15 is pulled toward the second end 13b of the cylindrical body 13, the fins 26a will be forced toward the inner wall of the cylindrical body 13, thus carrying out the stop and/or anti-removal function carried out by the stop element 26.

Furthermore, the fact that the stop element 26 is thus shaped allows it to adapt to variable inner diameters of the cylindrical body 13 and/or of the first syringe element 12. Naturally, the diameter of the stop element 26 may vary depending on the inner diameter and/or capacity of the cylindrical body 13 of the first syringe member 12.

The larger diameter of the stop element 26 (determined by the free end of the fins 26a) is slightly larger than the diameter of the capsule or gasket 20 and/or of the head 15a of the plunger 15. In any case, given the fins 26a are flexible, the larger diameter of the stop element 26 is variable.

The flexibility of the stop element 26 may be given by the presence of the space or gap 26b between one fin and the other, or by the flexibility of the material with which the fins 26a are made, or by a combination thereof.

As observable in the FIGS. 11A and 11B, the plurality of fins 26a is substantially rectangular parallelepiped-shaped, possibly with bevelled edges or with the free end thereof substantially rounded or curved, but other shapes can be provided. They are arranged substantially along the circumference of the capsule or gasket 20, for example to form a ring of fins 26a. In this manner, they project substantially from the capsule or gasket 20 and/or from the head 15a of the plunger 15.

Furthermore, there is present an annular hollow area 26c arranged between the plurality of fins 26a and the head 15a of the plunger 15. Such annular hollow area 26c allows the movement of the fins 26a toward the head 15a of the plunger 15, so as to allow the anti-removal function of the stop element 26.

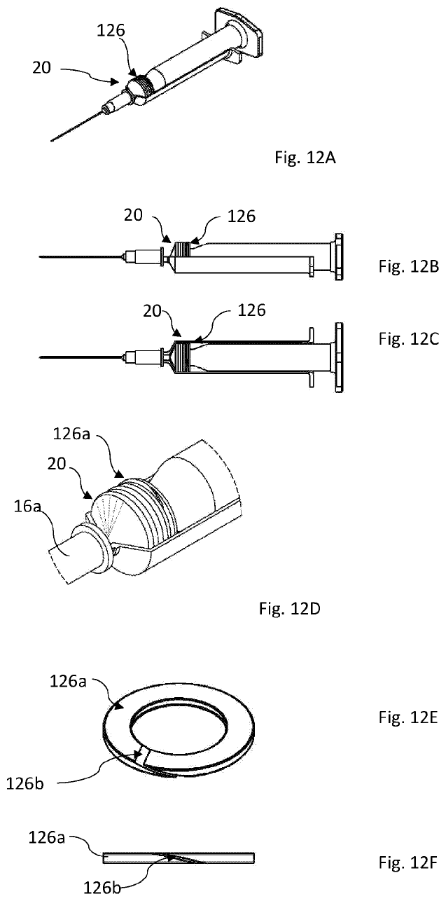

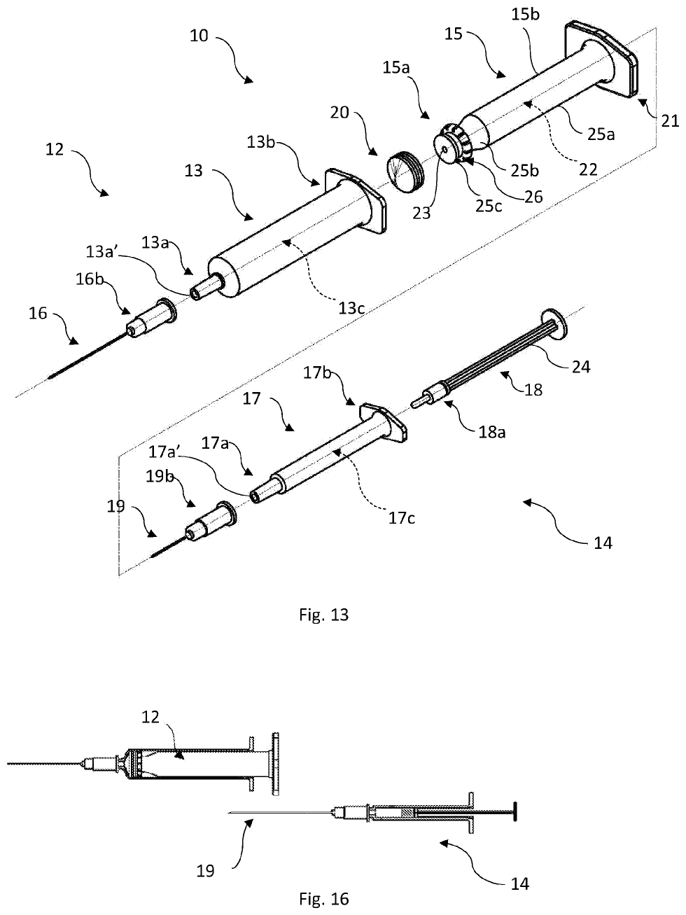

FIGS. 12A, 12B, 12C and 12D show a different version of the stop element 126.

It has the same function as the stop element 26 but shaped to form a ring nut 126a.

The ring nut 126a is substantially annular-shaped with a circular cross-section (visible in FIGS. 12E and 12F). It is housed between the capsule or gasket 20 and the head 15a of the plunger 15 and/or it is fitted at the apical area of the cone or truncated cone portion 25b of the stem 15b of the plunger 15.

The ring nut 126a of the stop element 126 has a cut 126b, for example transverse and/or inclined, as shown in FIG. 12F.

In this manner, the ring nut 126a has an open-ring configuration and/or it can be adapted to variable inner diameters of the cylindrical body 13 of the first syringe element 12.

Naturally, the diameter of said ring nut 126a may vary according to different capacities and/or different inner diameters of the first syringe element 12.

As indicated also for the fins 26a of the stop element 26, the ring nut 126a allows the sliding of the plunger 15 toward the first end 13b of the cylindrical body 13 while, when the opposite movement of the plunger 15 away from the first end 13c occurs, the stop element 126 prevents the removal of the piston 15 from the cylindrical body 13 thanks to the ring nut 126a. As a matter of fact, the cut 126b determines a variation in the overall diameter of the ring nut 126a following the movement of the plunger 15 (a diameter which decreases while the plunger 15 is step for pushing the plunger and which increases during the step for removing the piston, actually preventing the plunger from exiting from the cylindrical body 13).

In some embodiments, the stop element 26, 126 is positioned distally with respect to the capsule or gasket 20, that is, arranged between the capsule or gasket 20 and the stem of the plunger 15.

Therefore, as observable from the description above, in order to carry out the desired function, the second syringe element 14 is coaxial with the first syringe element 12, and/or with the internal channel 22 of the stem 15b of the plunger 15 of the first syringe element 12. Similarly, the direction of insertion of the second needle 19 at the first needle 16 occurs coaxially, so as to allow the injection of the substance contained in the second syringe element 14 exactly at the point of injection of the first needle 16 of the first syringe element 12.

The coaxiality and/or the presence at least of the hollow internal channel 22 of the stem 15b of the plunger 15 thus ensures the centring of the second needle 19.

The at least one substance present in the inner chamber which is formed in the cavity 13c between the cylindrical body 13 and the plunger 15 when the latter is at least partially housed in the cylindrical body 13 (first chamber) and/or which is formed in the space 17c between the cylindrical side wall 17 and the plunger 18, when the latter is housed at least partially in the cylindrical side wall 17 (second chamber), in some embodiments, it is inserted into the respective first syringe element 12 and/or second syringe element 14 immediately before use.

For example, as a matter of fact, the surfactant must form a sort of foam that is formed before the injection thereof. Similarly, cyan acrylic glue (Loctite-Attak) or other adhesive agent polymerizes rapidly in contact with moisture and therefore it can only be loaded into the second syringe element 14 just before the use thereof.

In order to allow operation of the kit, in at least one version (not illustrated in the figures), the first syringe element 12 may comprise a pierceable membrane, arranged at the first end 13a of the cylindrical body 13. Such pierceable membrane is suitable to be pierced by the second needle 19 of the second syringe element 14 so that the second medical substance can be injected into the injection site of the first needle 16.

Furthermore, such pierceable membrane is suitable to allow the through-flow of a fluid (for example of the first medical substance, if present) only in the extrusion or injection direction, and not in the opposite suctioning direction, in order to prevent the second medical substance, in the injection step, from leaking in sites other than injection sites.

In some embodiments, there is at least one cap for covering and protecting the first needle 16 and/or the second needle 19.

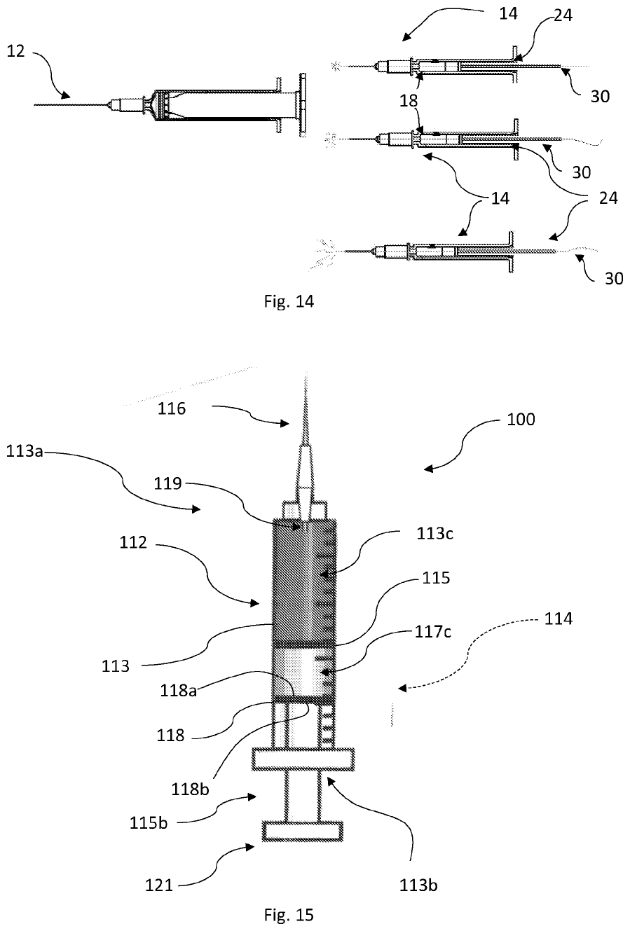

As observable in FIG. 14, a further version of the kit provides for that the first syringe element 12 (substantially shaped as described above) contains a substance to be injected into the human body while the second syringe element 14 comprises at least one element 30. In this case, the stem 24 of the piston 18 is hollow and it allows the insertion and housing of the element 30 (in the form of the devices listed above) thereinto. For example, FIG. 14 shows a second component 14 (shown in the upper position and to the right of the figure) which comprises an element 30 in the form of optical fibre and/or laser, suitable for example to heat and/or convey heat to the point of injection of the human body, while also visible is another second component 14 (shown in a central position and to the right of the figure) which comprises an element 30 in the form of a pipe for conveying steam and/or heat, or a second component 14 which comprises an element 30 in the form of a radio-frequency device or antenna (shown in a lower position and to the right).

The at least one element 30 has an extended shape and a thickness or section that is very small in size (with respect to the length thereof) so as to be able to pass through the second needle 19 and/or the first needle 16.

This version of the kit is useful, for example, when there arises the need to convey heat and/or moisture in the form of steam to the site of injection of the first substance (for example glue or an adhesive substance), or when there arises the need to dispense another sealing agent different from the first dispensed substance in such point.

In this manner, the second syringe element 14 acts as a carrier for the at least one element 30.

The at least one element 30, such as for example the laser fibre or the steam pipe illustrated in FIG. 14, exploits the coaxiality of the system to carry for example heat (laser or steam) exactly to the desired point.

As observable in FIG. 14, the at least one element 30 is inserted and/or housed at least partially inside the stem 24 of the piston 18, passing through the second needle 19 to reach near the first needle 16 (when the second syringe element 14 is housed inside the first syringe element 12) of the first syringe element 12, in which it can only be partially inserted and/or housed.

In order to do this, the second needle 19 pierces the plunger 15 and/or the capsule or gasket 20 of the first syringe element 12 as described above, so as to allow the at least one element 30 to reach in proximity of the first needle 16. Alternatively or additionally, the second needle 19 passes through the gap or hole 23 present in the plunger 15. Only after the second needle 19 has passed through the plunger 15, the at least one element (in the form of fibre or pipe) is pushed toward the first end 13a of the cylindrical body 13, passing through the second needle 19 and at least partially the first needle 16. Once in proximity of the site of injection, the at least one element 30 can carry out the function thereof, such as for example that of conveying heat which, in at least one application version, may have a thrombosing property with respect to the walls of the vein to be treated.

In order to be able to reach and pass beyond the second needle 19, the at least one element 30 is capable of in turn piercing the head 18a of the piston 18 of the second syringe element 14 (for example through a piercing element arranged at the inner end of the at least one element 30), or to pass through a hole or channel (possibly initially closed by a breakable membrane which is suitably positioned and which can be perforated by the at least one element 30) which is purposely provided at the head 18a of the piston 18.

In use, the concepts from the disclosure also provides for a method for the use of kit 10 and/or for the injection and/or insertion of at least one substance and/or at least one element comprising the following steps: providing a first syringe element 12 and at least a second syringe element 14, providing for at least one substance and inserting or loading at least one substance into the first syringe element 12 and/or the at least one second syringe element 14.

There are then steps of positioning the first syringe element 12 in use at an injection site of the human body, such as for example a varicose vein to be treated, injecting at least one substance (or first substance) at the injection site by the first syringe element 12 or suctioning a fluid, for example biological, from the injection site using the first syringe element 12.

Then there follows the step of positioning the plunger 15 to the end-stroke position at the first end 13a of the cylindrical body 13 of the first syringe element 12 (possibly injecting the substance contained therein into the injection site) and by a step of inserting the second syringe element 14 at the hollow internal channel 22 of the stem of the plunger 15 of the first syringe element 12. Such insertion occurs along a direction coaxial to the cylindrical body 13 and/or to the first syringe element 12 and/or to the internal channel 22 of the stem 15b of the plunger 15.

This insertion occurs so that the second needle 19 of the second syringe element 14 penetrates at least partially into the first end 13a of the cylindrical body 13 and/or into the first connection element 16b of the first needle 16.

At this point, the at least one substance loaded into the second syringe element 14 (or second substance) can be loaded by means of the first needle 16 of the first syringe element 12.

Should the first syringe element 12 suction a liquid or fluid present at the injection site, firstly there will be inserted a second syringe element 14 to suction the liquid or fluid suctioned and present inside the cylindrical body 13 of the first syringe element 12. Once this step has been completed, a further second syringe element 14 will be inserted, so as to proceed with the injection of the at least one substance loaded into the latter in the injection site.

The piston 18 of the second syringe element 14 is then carried and/or pushed in the direction of the second needle 19.

Alternatively, should a biological liquid be suctioned from a site of the human body, the method provides for suctioning said liquid by means of the first syringe element 12, at least partially removing the plunger 15 from the cylindrical body and thus creating a vacuum inside the cavity 13c so as to suction the biological liquid in question in use. Then, there is provided a second syringe element 14 having a second needle 19 (as shown in FIG. 16) with dimensions suitable to reach in proximity of the first needle 16 (despite the plunger 15 being in a position moved away with respect to the first end 13a of the cylindrical body 13) so as to be able to inject the substance contained in the second syringe element 14 into the suctioning site carried out by means of the first needle 16. Otherwise, there may be provided for the steps of connecting a system, for example a suctioning system, to the first syringe element 12 so as to eliminate the liquid suctioned by the first needle 16 from the first syringe element 12, thus so as to be able to approach the head 15a of the plunger 15 to the first end 13a of the first syringe element 12, and thus allow the second needle 19 to reach in proximity of the first needle 16 and carry out the injection of the substance contained in the second syringe element 14.

Naturally, in order for the kit to operate, the cylindrical body 13 of the first syringe element 12 has a length equal to or smaller than that of the second syringe element 14, so that the piston 18 can be actuated from the external by the operator when the plunger is fully inside the cylindrical body 13 and when the second syringe element 14 is positioned in the plunger 15 (as observable in the FIGS. 6A and 6B).

During the step for inserting the second syringe element 14, the second needle 19 pierces the plunger 15 (and/or a part thereof and/or the head 15a thereof and/or the pierceable membrane) and/or the capsule or gasket 20 and/or the through-passing of the second needle 19 through the gap or hole 23 of the plunger 15.

With reference to FIG. 15, a kit for the subsequent injection of substances into the human body according to a further version is indicated in its entirety with reference number 100. The elements corresponding to those of the previous version will be indicated with the same reference number, increased by one hundred.

With respect to the previous version, the kit 100 requires the components to be assembled together, so as to provide a compact and fast-to-use solution. Furthermore, this is a version that is still easy and simple to handle, which can also be used by non-specialized personnel.

The kit 100 comprises a first syringe element 112. With respect to the previous version, it is as if the first syringe element 12 housed the second syringe element 14 stably and permanently therein, for example in a constrained manner It can therefore be said that the kit 100 also comprises a second syringe element 114. As mentioned, the latter is stably and permanently constrained to the first syringe element 112.

The first syringe element 112 contains—therein—at least two substances, such as a first medical substance, such as for example a biocompatible surfactant substance, and a second medical substance, such as a biocompatible adhesive agent, of the type indicated above, or the same medical substance, but present in two different doses or densities. In particular, the first syringe element 112 comprises, as indicated for the first version, an internally hollow cylindrical body 113, a plunger 115 (or first piston 115) and a first needle 116.

The plunger 115 hermetically slides inside the cylindrical body 113 along a longitudinal direction specifically determined by the cylindrical body 113.

In particular, the plunger 115 has a head 115a, which, in use, is suitable, when fully inserted into the cavity 113c and/or into the cylindrical body 113, to internally come into contact with the first end 113a of the cylindrical body 113.

The head 115a is faced toward the first needle 116 or toward the second needle 119.

The second syringe element 114 comprises a piston 118 (or second piston 118) and a second needle 119.

The second syringe element 114 shares the cylindrical body 113 with the first syringe element 112. Therefore, unlike the second syringe element 14, the second syringe element 114 does not comprise a wall of its own, but substantially it consists of a piston 118 and a second needle 119. There is present a stem 115b for actuating the plunger 115 and the piston 118, which can be said to belong both to the first syringe element 112 and to the second syringe element 114.

Therefore, the kit 100 substantially consists of first cylindrical body 113, plunger 115, first needle 116, piston 118, second needle 119, stem 115b, obviously besides the at least one medical substance contained therein (besides the sealing and connection elements described hereinafter).

Inside the first syringe element 112 (and/or the cylindrical body 113), the presence of the plunger 115 and the piston 118 determines the definition of two spaces or chambers in which the two medical substances, such as in detail a first cavity 113c and a space 117c, can be housed.

In detail, the first cavity 113c is arranged between the plunger 115 and a first end 113a of the cylindrical body 113, while the space 117c is arranged between the plunger 115 and the piston 118.

In detail, the first medical substance is present in the first cavity 113c while the second medical substance is present in the space 117c.

Therefore, the piston 118 has a head 118a in contact with the second medical substance and a base face 118b, in use external and opposite with respect to the head 118a.

Connected in use to the base face 118b is the stem 115b which is suitable to be actuated so as to allow the dispensing of the at least one medical substance or both medical substances, when both the first and second medical substances are present.

The first needle 116 and the second needle 119 are internally hollow and/or comprise respectively a first inner lumen and a second inner lumen, possibly placed in fluid communication and/or possibly coaxial.

Then there may be present a first connection element between the first needle 116 and the first end 113a of the cylindrical body 113 and/or a second connection element between the second needle 119 and the first end 113a of the cylindrical body 113 to connect, possibly removably, the first needle 116 and/or the second needle 119 to the cylindrical body 113.

In greater detail, the first needle 116 is connected externally to the first end 113a of the cylindrical body 113 while the second needle 119 is connected internally to said first end 113a. Therefore, the first needle 116 and the second needle 119 have an opposite orientation.

The cylindrical body 113 has a second end 113b, opposite the first end 113a, and which constitutes the open end of the cylindrical body 113.

The plunger 115 slides inside the cavity 113c while the piston 118 slides inside the space 117c but actually also, in a first operative step, inside the first cavity 113c.

The first end 113a has—therein—a connection channel with the first lumen of the first needle 116 and with the second lumen of the second needle 119, as well as with the cavity 113c.

In this further version, the stem 115b may be hollow or solid.

In use, the operator acts on the stem 115b, acting in thrust thereon.

Therefore, the stem 115b is pushed toward the first needle 116, actually determining the outflow of the at least one first medical substance housed in the cavity 113c. Since the space 117c is actually closed and filled with the at least one second medical substance, it is incompressible. When the stem 115b is actuated, it pushes the piston 118 and the space 117c, which in turn pushes the plunger 115 toward the first end 113a of the first syringe element 112. Thus, the piston 118 and the plunger 115 slide as a single body until the plunger 115 reaches in proximity of the first end 113a of the cylindrical body 113.

This is a first operative step of using the kit 100.

Once the first medical substance has been fully or substantially fully dispensed, the plunger 115 is at the inner part of the first end 113a of the first syringe element 112. In this manner, the second needle 119, which is arranged inside the first end 113a, is capable of piercing the plunger 115.

Therefore, actually while the tip of the first needle 116 is external to the first syringe element 112, for dispensing the at least one medical substance at the site of interest of the human body, the second needle 119 has a tip facing toward the internal of the cylindrical body 113. in particular, toward the plunger 115.

The length of the second needle 119 is therefore greater than the thickness of the plunger 115, so as to be able to pierce it and to be able to penetrate into the space 117c.

As the plunger 115 approaches the second needle 119, the latter begins the piercing of the plunger 115.

After piercing plunger 115, given that the space 117c is no longer a closed space, still pushing on the stem 115b, allows to move the piston 118 toward the plunger 115 and the dispensing of the second medical substance contained in the space 117c. Therefore, in such second operative step the second medical substance is dispensed exactly into the site of injection of the first medical substance.

The second medical substance is dispensed, through the second needle 119, by means of the first needle 116.

In this case, the stem 115b is almost fully housed inside the cavity 113c, except for the end part 121 part.

The plunger 115 and/or the head 115a of the plunger 115 may comprise a capsule or gasket, as indicated previously for the kit 10.

Such capsule or gasket is hermetically-sealed in the sense that it allows the sliding of the plunger 115 inside the cavity 113c of the cylindrical body 113 but, at the same time, it prevents the medical solution contained in the cavity 113c from flowing out from the side of the capsule or gasket. In which case, the second needle 119 pierces the capsule or gasket of the plunger 115.

In this version, the kit is filled with at least one substance at the factory and not by the physician.

A similar capsule or gasket may also be provided for the piston 118.

The embodiments described in this disclosure may operate thanks to the presence of the second needle 19, 119 which is suitable to pass through the plunger 15, 115 in use.

In some embodiments, the plunger 115 may comprise a stop element, as described for the kit 10.

It has thus been observed that the concepts of the disclosure may adapt to specific surgical requirements, by providing a kit that is easy and practical to use, which allows precision at the implantation site even for injections subsequent to the first, at the same injection site.

Characteristics described for an embodiment or variant may also be present in other embodiments or variants, without departing from the scope of protection outlined by the attached claims.

The kit 10, 100 according is susceptible to numerous modifications and variants without departing from the scope of protection of the claims that follow.

In accordance with common practice, the various features illustrated in the drawings may not be drawn to scale. The illustrations presented in the present disclosure are not meant to be actual views of any particular apparatus (e.g., device, system, etc.) or method, but are merely idealized representations that are employed to describe various embodiments of the disclosure. Accordingly, the dimensions of the various features may be arbitrarily expanded or reduced for clarity. In addition, some of the drawings may be simplified for clarity. Thus, the drawings may not depict all of the components of a given apparatus (e.g., device) or all operations of a particular method.

Terms used herein and especially in the appended claims (e.g., bodies of the appended claims) are generally intended as “open” terms (e.g., the term “including” should be interpreted as “including, but not limited to,” the term “having” should be interpreted as “having at least,” the term “includes” should be interpreted as “includes, but is not limited to,” etc.).

Additionally, if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations.

In addition, even if a specific number of an introduced claim recitation is explicitly recited, it is understood that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” or “one or more of A, B, and C, etc.” is used, in general such a construction is intended to include A alone, B alone, C alone, A and B together, A and C together, B and C together, or A, B, and C together, etc. For example, the use of the term “and/or” is intended to be construed in this manner

Further, any disjunctive word or phrase presenting two or more alternative terms, whether in the description, claims, or drawings, should be understood to contemplate the possibilities of including one of the terms, either of the terms, or both terms. For example, the phrase “A or B” should be understood to include the possibilities of “A” or

“B” or “A and B.”

Additionally, the use of the terms “first,” “second,” “third,” etc., are not necessarily used herein to connote a specific order or number of elements. Generally, the terms “first,” “second,” “third,” etc., are used to distinguish between different elements as generic identifiers. Absence a showing that the terms “first,” “second,” “third,” etc., connote a specific order, these terms should not be understood to connote a specific order. Furthermore, absence a showing that the terms “first,” “second,” “third,” etc., connote a specific number of elements, these terms should not be understood to connote a specific number of elements. For example, a first widget may be described as having a first side and a second widget may be described as having a second side. The use of the term “second side” with respect to the second widget may be to distinguish such side of the second widget from the “first side” of the first widget and not to connote that the second widget has two sides.

All examples and conditional language recited herein are intended for pedagogical objects to aid the reader in understanding the invention and the concepts contributed by the inventor to furthering the art, and are to be construed as being without limitation to such specifically recited examples and conditions. Although embodiments of the present invention have been described in detail, it should be understood that the various changes, substitutions, and alterations could be made hereto without departing from the spirit and scope of the present invention.

1. A kit for injection of at least one substance in a fluid or liquid state and/or for insertion of at least one substance and at least one element in an injection or insertion site of a human body, the kit comprising:
a first syringe element; and
at least one second syringe element,
wherein said first syringe element comprises:
an internally hollow cylindrical body,
a plunger; and
a first needle, in which said plunger is adapted in use to slide inside said cylindrical body,
wherein said at least one second syringe element comprises:
a piston; and
a second needle,
wherein the at least one substance and/or said at least one element is inserted inside said cylindrical body and/or inside said second syringe element and said second needle is adapted in use to pass through said plunger.

2. The kit according to claim 1, wherein said plunger comprises a head, facing, in use, said first needle and a stem, internally hollow and/or comprising in its inner part an internal channel, and/or wherein said kit comprises a stem.

3. The kit according to claim 2, wherein said first syringe element is adapted in use to at least partially house in its inner part said at least one second syringe element and/or in which said stem and/or said internal channel of said stem is adapted in use to at least partially house said at least one second syringe element inside the internal channel.

4. The kit according to claim 2, wherein said internal channel is coaxial with said stem and/or with said first syringe element and/or wherein said internal channel is shaped in such a way as to allow a housing of said second syringe element inside the internal channel and/or wherein said internal channel has a conformation corresponding and/or complementary to that of said second syringe element, but larger to allow the, removable, housing of said second syringe element.

5. The kit according to claim 2, wherein said plunger and/or said stem comprises a substantially tubular shaped portion and, at said head, a cone or truncated cone portion, in which said channel of said stem involves both said substantially tubular shaped portion and said cone or truncated cone portion.

6. The kit according to claim 10, wherein at an apical area of said cone or truncated cone portion, facing said first needle, there is a discoidal portion, which acts as a base of support and constraint for said capsule or gasket.

7. The kit according to claim 1, wherein said first needle and said second needle are internally hollow and/or comprise respectively a first internal lumen and a second internal lumen through which the at least a medical substance can in use flow.

8. The kit according to any one of the preceding claims claim 2, wherein said cylindrical body comprises a first end to which said first needle is constrained, possibly in a removable way, a cavity inside said cylindrical body and a second end, opposite to said first end, wherein said second end is open and for access to said cavity.

9. The kit according to claim 8, wherein said second syringe element comprises a cylindrical side wall, internally hollow, comprising a tip, to which said second needle is constrained, in a removable way, an internal space to said cylindrical side wall for housing said at least one substance, and a distal end, opposite to said tip, wherein said distal end is open and for access to said space, in which said piston is adapted in use to slide inside said cylindrical side wall.

10. The kit according to claim 2, wherein said plunger and/or said head of said plunger comprises a capsule or gasket, wherein said capsule or gasket is made of rubber and/or has a substantially conical conformation or corresponding to an internal zone of said first end of said cylindrical body, and/or in which said capsule or gasket is adapted in use to be perforated by said second needle.

11. The kit according to claim 6, wherein, at said capsule or gasket, said head and/or said plunger and/or said discoidal portion and/or said cone or truncated cone portion has an opening or hole for passage in use of said second needle.

12. The kit according to claim 9, comprising a first connection element between said first needle and said cylindrical body to connect the cylindrical body, in a removable way, to said first needle and/or a second connection element between said second needle and said cylindrical body or said cylindrical side wall to connect the cylindrical body, in a removable way, to said second needle and/or in which said plunger comprises an opening or a hole at said head, in which said first connection element is adapted in use to house at least partially said second needle.

13. The kit according to claim 1, wherein said at least one substance is a medical substance such as a biocompatible surfactant substance, such as for example a surfactant sclerosing drug such as sodium tetradecyl sulfate known by the trade name Fibro-Vein® or Trombovar® or oxy-polyethoxy dodecane known by the trade name Atossisclerol® or Sotradecol®, etc., a biocompatible gluing agent, for example based on cyanoacrylate, such as for example the one known by the trade name Loctite-Attak, and/or a biocompatible cementing element.

14. The kit according to claim 10, comprising a stop element, of substantially annular conformation, adapted in use to at least partially prevent extraction of said plunger from said cylindrical body, wherein said stop element is placed at said capsule or gasket and/or said head of said plunger.

15. The kit according to claim 14, wherein said stop element comprises a plurality of fins, flexible if desired, arranged annularly around said head and/or wherein each fin departs from said head outwards with respect to said plunger and/or facing in use an end part of said plunger and/or said second end of said cylindrical body and/or wherein each fin is separated from each adjacent fin by a space or gap between one fin and the other, so that each fin of said plurality flexes and/or moves and/or changes position if said plunger is moved towards said first end or towards said second end of said cylindrical body.

16. The kit according to claim 15, wherein said fins have a free end and said stop element has a larger diameter at said free end which is slightly greater than the diameter of said capsule or gasket and/or of said head of said plunger and/or wherein there is an annular hollow area located between said plurality of fins and said head of said plunger for allowing bending and/or movement of said fins towards said head.

17. The kit according to claim 14, wherein said stop element has a substantially annular ring nut conformation with cylindrical section and/or has a ring nut conformation comprising a cut so that said ring nut is adaptable with variable internal diameters of said cylindrical body of said first syringe element.

18. The kit according to claim 9, wherein said at least one substance is housed in a first internal chamber formed in said cavity between cylindrical body and plunger when the cylindrical body is at least partially housed inside said cylindrical body of said first syringe element and/or in a second internal chamber formed in said space between cylindrical side wall and piston, when the cylindrical body is at least partially housed inside said cylindrical side wall of said second syringe element and/or in a second internal chamber formed between said plunger and said piston when both are housed inside of said cylindrical body of said first syringe element (112).

19. The kit according to claim 1, wherein said second syringe element comprises at least one element, in the form of an optical fiber device, a laser fiber device, a device capable of transporting and/or delivering a gas, a radiofrequency device or antenna, for example having a device capable of generating heat and/or radiation, wherein said at least one element has an elongated conformation and is able to be housed inside said piston , said second needle and to reach near or be at least partially housed in said first needle and/or wherein said at least one element possibly comprises a perforating element placed at an internal part thereof to perforate said piston and/or wherein said at least one element is able to be housed in said hollow stem of said piston and to be housed or pushed into a channel present in said head of said piston.

20. The kit according to claim 1, wherein said first syringe element comprises a pierceable membrane, located at said first end of said cylindrical body, in which said pierceable membrane is adapted to be pierced by said second needle of said second syringe element so that said second needle comes in proximity of said first needle..

21. The kit according to claim 1, wherein said first needle is facing in use outside of said first syringe element and/or towards the injection site while said second needle is facing in use a first cavity of said cylindrical body, in which said first needle and said second needle are coaxial.

22. (canceled)

23. (canceled)

24. (canceled)

25. (canceled)

26. (canceled)

27. (canceled)

28. (canceled)
